(19) United States
(12) Patent Application Publication
(10) Pub. No.: US 2025/0318792 A1
(43) Pub. Date: Oct. 16, 2025

(54) DIGITAL MOBILE RADIOGRAPHY SYSTEMS AND METHODS

(71) Applicant: RAD PHYSICS, INC., Vestavia Hills, AL (US)

(72) Inventor: Gary T. Barnes, Vestavia Hills, AL (US)

(73) Assignee: RAD PHYSICS, INC., Vestavia Hills, AL (US)

(21) Appl. No.: 18/764,236

(22) Filed: Jul. 4, 2024

Related U.S. Application Data
(63) Continuation-in-part of application No. 18/520,857, filed on Nov. 28, 2023.

(51) Int. Cl. A61B 6/42; A61B 6/00; A61B 6/04; A61B 6/06

(52) U.S. Cl. CPC A61B 6/4291; A61B 6/0492; A61B 6/06; A61B 6/4405

(57) ABSTRACT

A mobile radiography system has an x-ray source assembly configured to activate a digital x-ray detector through X-rays striking the digital x-ray detector and one or more shutters installed in a collimator in a stream of the X-rays configured to project an alignment image onto the digital x-ray detector. Further, the mobile radiography system has a processor that receives data indicative of the alignment image and the anatomy of interest, generates a first digital image indicative of the alignment image and the anatomy of interest, and analyzes the first image and determining if a focal spot of the x-ray source assembly is aligned with a focal axis of an anti-scatter grid and the digital x-ray detector based on the first digital image.

### CROSS-REFERENCE TO RELATED APPLICATIONS

This application is a continuation-in-part and claims priority to U.S. patent application Ser. No. 18/520,857 entitled Digital Mobile Radiography Systems and Methods and filed on Nov. 28. 2023, which is incorporated herein by its entirety.

### BACKGROUND

A digital mobile radiography unit consists of a planar digital image detector, computer and display, x-ray source, manual x-ray beam collimator, x-ray generator and control, and supporting mobile assembly. A mobile radiographic exam consists of the operator moving the unit to the patient's bedside. The operator then positions the planar digital image detector beneath the anatomy of interest and the x-ray source above the anatomy of interest and visually aligns the source with the image detector. The operator also visually adjusts the x-ray beam collimation.

The operator selects the x-ray tube potential (kV) for the type of exam and the x-ray tube current—exposure time product (mAs) and initiates the x-ray exposure. The x-ray image is captured by the digital image detector and communicated wirelessly to the unit's computer, processed, and displayed for viewing by the operator. When an anti-scatter grid is used it is placed on the digital image detector prior to positioning the detector (and grid) beneath the anatomy of interest.

The detected x-ray image consists of two classes of X-rays, primary and secondary. The primary X-rays have travelled in the straight-line path from the x-ray tube focal spot, passing through the patient, to the image detector and carry anatomical information. Secondary X-rays have interacted with atoms and electrons in the patient, are scattered, and do not travel in a straight line from the focal spot to the image detector and carry no information. The secondary or scattered X-rays form an out-of-focus image superimposed on and degrading the quality of the primary x-ray image. An anti-scatter grid is a device that reduces the amount of scattered radiation reaching the detector. The operator positions the anti-scatter grid on the opposite side of the patient from the x-ray source and between the patient and the x-ray detector. The anti-scatter grid reduces the contribution of the secondary out-of-focus scattered X-rays and increases the image's contrast resolution, and consequently, the visibility of anatomical structures.

A grid consists of an array of radiopaque foil strips (usually lead) separated by strips of radiolucent interspace material (usually aluminum or fiber). Commonly used anti-scatter grids have their strips progressively tilted with increasing distance from the center of the anti-scatter grid and focused on a line above the center of the anti-scatter grid (grid focal axis). When the anti-scatter grid is properly positioned and aligned between the patient and image detector, the x-ray tube focal spot is on the focal axis of the anti-scatter grid, the anti-scatter grid is aligned, and the image-forming primary X-rays “see” only the edges of the lead foil strips and small fraction are absorbed; whereas, the scattered X-rays “see” a much greater area of lead and a large fraction are absorbed. Higher ratio grids control scatter better than low ratio grids but require more precise alignment. When a grid is misaligned a greater percentage of the primary X-rays are absorbed by the grid, and often non-uniformly, then when the grid is aligned. Conversely, the percentage of scattered X-rays transmitted by the grid is minimally affected by its misalignment. When a grid is misaligned, the contribution of scattered X-rays to the detected x-ray image is increased and the quality of the detected x-ray image is degraded when compared with an image obtained when the anti-scatter grid is properly aligned.

When an anti-scatter grid is employed in mobile radiography, it is aligned visually with the x-ray tube focal spot and the alignment achieved is not precise. As a result, anti-scatter grids are not used in exams where grid misalignment can mimic pathology. In exams where an anti-scatter grid misalignment does not mimic pathology, low ratio anti-scatter grids (6:1 or 8:1) are employed in which the misalignment effects are less than what occurs with a high ratio grid.

### DESCRIPTION OF THE FIGURES

The present disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 is a diagram of an exemplary mobile radiography system in accordance with an embodiment of the present disclosure.

FIG. 2 is a block diagram of an exemplary radiography computing device as shown in FIG. 1.

FIG. 3A is a diagram of an exemplary x-ray source assembly and collimator as shown in FIG. 1.

FIG. 3B is a diagram of exemplary collimator shutters for use in the collimator as shown in FIG. 1.

FIG. 3C is a diagram of an exemplary alignment image produced on a detector via the collimator shutters shown in FIG. 3B.

FIG. 4 is an exemplary articulated arm as shown in FIG. 1 in accordance with an embodiment of the present disclosure.

FIG. 5 is a diagram of a portion of an anti-scatter grid as shown in FIG. 1.

FIG. 6 is a diagram of a plurality of exemplary lead foil strips of an anti-scatter grid, as shown in FIG. 1, and corresponding focal axis showing misalignment and alignment of a focal spot of an x-ray source assembly with the focal axis of the anti-scatter grid.

FIG. 7A is an exemplary test image projection produced by X-rays traveling through the collimator by the shutters as shown in FIG. 3C.

FIG. 7B is a diagram of an exemplary position of the x-ray source as shown in FIG. 1 showing a focal spot of the x-ray source assembly in a misaligned position and an aligned position.

FIG. 8 is a flowchart depicting exemplary architecture and functionality of the radiography system as shown in FIG. 1.

FIG. 9 is a block diagram of an exemplary artificial intelligence implementation for use in the radiography computing device of FIG. 1.

### DETAILED DESCRIPTION

The present disclosure provides systems and methods to improve scatter control in digital mobile radiography with minimal effort by the operator. Improved scatter control is achieved with automated, rapid, and accurate alignment of a digital mobile radiographic unit's x-ray tube focal spot with a focal axis of an anti-scatter grid and a digital x-ray detector.

The present disclosure describes a digital mobile radiography unit with the features of an automatic x-ray beam collimation, high ratio (12:1) anti-scatter grid, and x-ray focal spot—image detector location and orientation measuring. The digital mobile radiography unit tracks the location of each degree of freedom of motion of the x-ray tube focal spot and, when directed, moves the x-ray tube focal spot so that the x-ray focal spot aligns with a focal axis of the anti-scatter grid, is centered on the digital X-ray detector, and collimates the spatial extent of an x-ray beam to a digital x-ray detector. The spatial extent of the x-ray beam includes the length and the width of the primary x-ray beam incident on the patient and the digital x-ray detector.

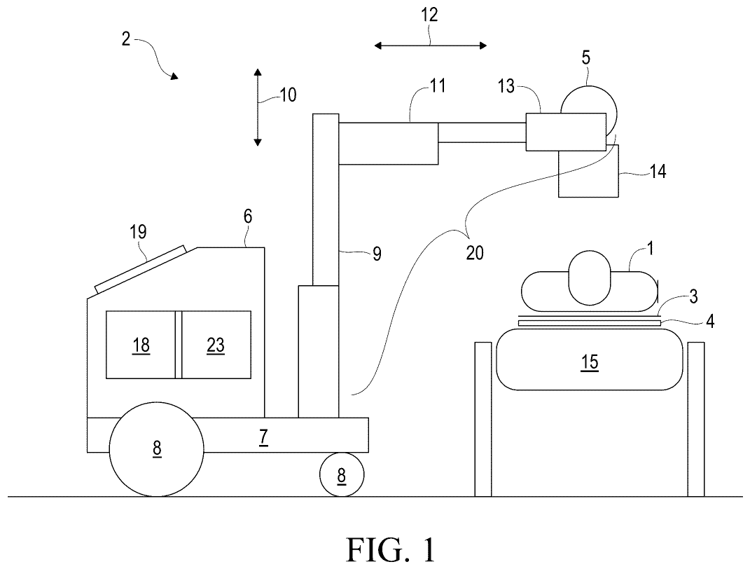

FIG. 1 is an exemplary mobile radiography system 2 in accordance with an embodiment of the present disclosure. The mobile radiography system 2 comprises a cabinet 6. Cabinet 6 houses a radiography computing device 18, an x-ray generator 23, and a display device 19. Note that in one embodiment there may be batteries (not shown) to power various components of the mobile radiography system 2.

Cabinet 6 is coupled to a base 7. Base 7 is coupled to one or more wheels 8 that allow the mobile radiography system 2 to be transported where needed in a medical facility for performing bedside radiography. The mobile radiography system 2 is transported using controls (not shown) by the operator (not shown) via motors (not shown) coupled to one or more wheels 8.

Base 7 is coupled to an articulated arm 20. In one embodiment, the articulated arm 20 comprises a vertical column 9. The vertical column 9 is telescoping allowing movement in a vertical direction indicated by the double arrow 10.

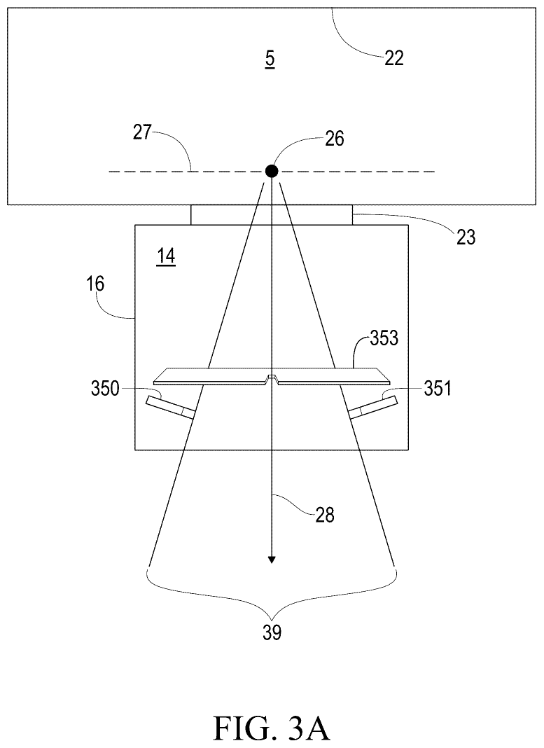

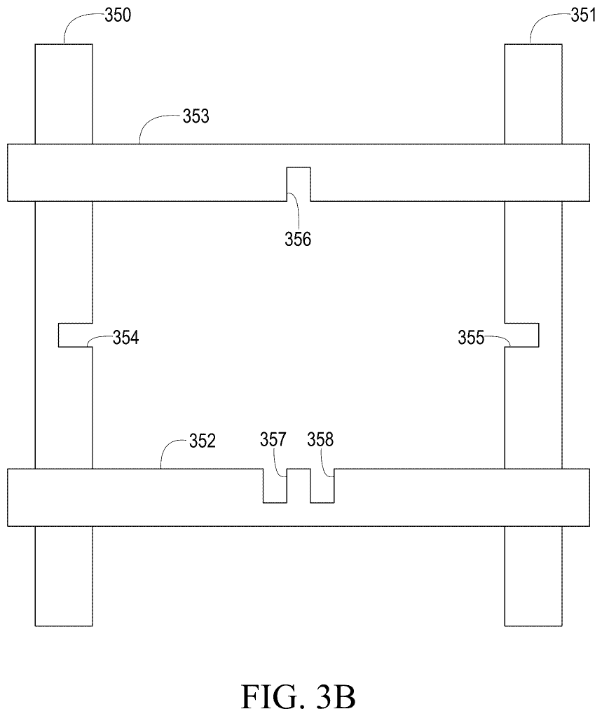

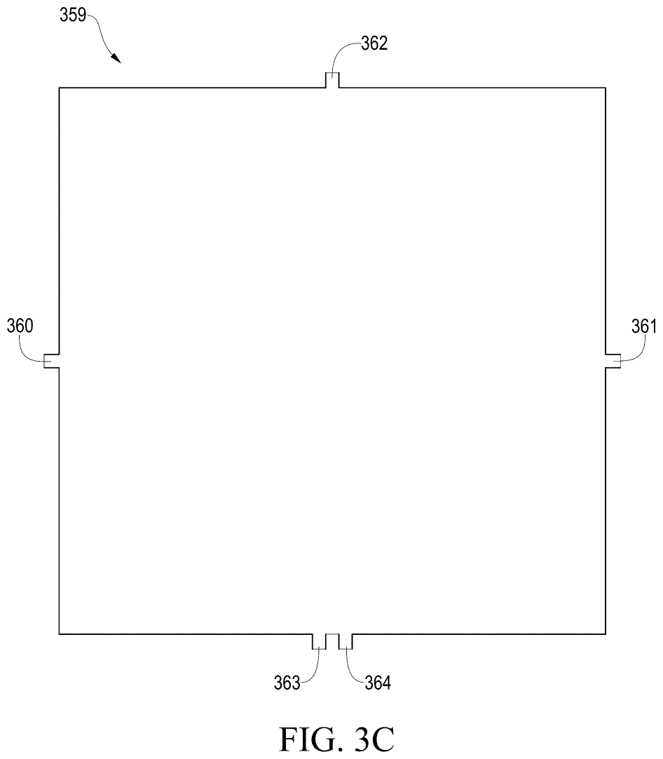

Further, the articulated arm 20 comprises horizontal arm 11. The horizontal arm 11 is coupled to the vertical column 9. In one embodiment, the horizontal arm 11 is telescoping allowing movement in a horizontal direction indicated by the double arrow 12. The articulated arm 20 further comprises a gimbal 13 and bracket (not shown) coupled to the horizontal arm 11 and gimbal 13. The bracket controls one or more degrees of motion freedom of the x-ray source assembly 5. Gimbal 13 is coupled to the x-ray source assembly 5 and controls one or more degrees of motion freedom of the x-ray source assembly 5. Also coupled to the x-ray source assembly 5 is a collimator 14. The collimator 14 is configured to spatially restrict the span of an X-ray beam emitted by the x-ray tube (not shown) contained in the x-ray source assembly 5. Moveably coupled within the collimator is one or more collimator shutters (not shown) in accordance with an embodiment of the present disclosure. The collimator shutters are further described with reference to FIG. 3A and FIG. 3B.

In operation the anti-scatter grid 3 and the digital detector 4 are positioned beneath the patient's anatomy of interest. The articulated arm 20 is moved manually by the operator, and the x-ray source assembly 5 is positioned. Further, the collimator 14 is adjusted so that the x-ray beam (not shown) is directed at the patient 1 and patient's anatomy of interest. In one embodiment, the patient 1 is placed on a bed 15. A digital x-ray detector 4 is placed behind the patient 1 to capture an anatomical area of interest. In one embodiment, the digital x-ray detector 4 comprises a flat-panel two-dimensional active-sensor digital matrix detector array. The digital x-ray detector 4 is configured to convert images (not shown) captured to digital data in real-time so that the images are available for analysis and viewing within seconds. In this regard, images captured are wirelessly communicated to the radiography computing device 18, and the radiography computing device 18 displays the images on the display device 19. A focused anti-scatter grid 3 is placed between the digital x-ray detector 4 and the patient. The anti-scatter grid 3 limits the amount of scattered radiation reaching the digital x-ray detector 4, which improves the quality of diagnostic x-ray images. Reducing the scattered X-rays increases the final image's contrast resolution, and consequently the visibility of anatomy. In one embodiment, the anti-scatter grid 3 is a high ratio (;::;12:1) anti-scatter grid. The radiography computing device 18 monitors the movement of the articulated arm 20 and tracks the different degrees of motion freedom of the x-ray focal spot 26 and its location. In addition, the radiography computing device 18 determines the x-ray detector location and orientation relative to the x-ray focal spot 26 and central ray 28.

Further, the radiography computing device 18 tracks the collimator settings and x-ray beam dimensions. Through operator cues or automatically, the radiography computing device 18 moves the x-ray tube focal spot to a desired location.

The automatic collimator 14 allows for automatic or manual adjustment of the x-ray beam size and when directed automatically adjusts the length and width of the x-ray beam to desired dimensions.

In operation, X-rays emitted from the x-ray source assembly 5 are directed through the collimator such that the X-rays in the X-ray beam defined by the collimator shutters pass through the collimator and are incident on the patient 1. The X-rays passing through the patient 1 are imaged on the digital x-ray detector 4. A configuration of radiopaque fiducial markers in present in the collimator shutters determines a test image that is detected by the digital x-ray detector 4. At a typical source-to-image detector distance (SID) the image of the test pattern is smaller (i.e., 15 cm×15 cm) than a 35 cm×43 cm or 43 cm×43 cm digital x-ray image detector 4.

In examining the patient 1 with the mobile radiography system 2, an operator (not shown) moves the mobile radiography system 2 to the patient's bedside and positions the digital x-ray detector 4 and anti-scatter grid 3 beneath the patient's anatomy of interest. The operator moves the x-ray source assembly 5 above the patient and visually (manually) aligns the x-ray source assembly 5 with the digital x-ray detector 4 and anti-scatter grid 3.

The collimator 14 is then adjusted. In this regard, adjustment of the collimator 14 is effectuated so that the X-ray beam defined by the collimator shutters and incident on the detector is a fraction of the area of the detector. In one embodiment, the collimator 14 automatically adjusts the x-ray beam size via adjustment of the collimator shutters. After the shutters are adjusted, a high kV, low dose x-ray image of a pattern created by the collimator shutters and patient's anatomy of interest is captured and communicated wirelessly to the radiography computing device 18. Note that in another embodiment, the radiography computing device 18 may direct the operator to manually adjust the collimator 14. The operator then initiates image exposure.

The radiography computing device 18 automatically moves or directs the operator to move the collimator shutters a sufficient amount via the display device 19. Thereafter, a second high kV, low dose slightly larger x-ray image of the collimator shutters over the patient's anatomy of interest is captured by the digital x-ray detector 4 and communicated wirelessly to the radiography computing device 18. The radiography computing device 18 subtracts the second image from the first image leaving only the image of the test pattern (the “test pattern image”) and removing the obfuscating effects of the patient's anatomy.

The radiography computing device 18 analyzes the test pattern image captured and determines the tilt of the plane of the digital x-ray detector 4, the plane of the anti-scatter grid 3, the location of the center of the digital x-ray detector 4, and the length and width orientation of the digital x-ray detector 4 relative to the location of and degrees of movement freedom of the x-ray focal spot.

The radiography computing device 18 communicates to the operator, e.g., via the display device 19, notice that the information has been communicated. Under operator control, the radiography computing device 18 automatically moves the x-ray focal spot so that it lies on the focal axis of the anti-scatter grid 3, and the central X-ray is centered on and orthogonal to the planes of the anti-scatter grid 3 and digital x-ray image detector 4.

Note that in one embodiment, the radiography computing device 18 directs the operator to manually move the articulated arm 20. The directions for moving the articulated arm 20 are provided such that movement of the articulated arm 20 aligns the x-ray focal spot so that it lies centrally on a focal axis of the anti-scatter grid 3. Note that in one embodiment, the computing device 18 automatically locks the articulated arm 20 in the different degrees of freedom when the x-ray focal spot is aligned after movement by the operator.

The collimator 14 automatically collimates the x-ray beam to the active area of the digital x-ray detector 4 via the collimator shutters. Note that the collimator 14 may also be collimated manually by the operator in other embodiments.

Further, the radiography computing device 18 moves and aligns each degree of the articulated arm 20 and locks each degree of freedom of the articulated arm 20 so that the x-ray focal spot of the x-ray source assembly 5 is in alignment with the anti-scatter grid focal axis and centered on the digital x-ray detector 4. When alignment of each of the degrees of freedom of the articulated arm 20 are achieved and the x-ray beam is centered on and collimated to the digital x-ray detector 4, the radiography computing device 18 notifies the operator, e.g., via the display device 19. Upon notification, the operator selects the x-ray techniques for the exam and patient's habitus and initiates the x-ray exposure. The digital x-ray detector 4 transmits data indicative of the resultant image data 36, obtained with a properly aligned high ratio grid and good scatter control, to the radiography computing device 18 for processing and display via the display device 19.

In one embodiment, the radiography computing device 18 directs the operator to sequentially move one or more degrees of freedom of the articulated arm 20 at a time and the motion of that degree of freedom is locked when its desired location is achieved. When the alignment of all the degrees of freedom has been achieved and the x-ray beam is centered on and collimated to the digital x-ray detector 4, an indication is given to the operator. The operator selects the x-ray techniques for the exam and patient's habitus and initiates the x-ray exposure. The resultant image, obtained with a properly aligned high ratio grid and good scatter control, is communicated wirelessly to the radiography computing device 18 for processing and display.

For a given digital x-ray detector 4 and anti-scatter grid 3, the patient's anatomy of interest and habitus determines the x-ray tube potential (kV) and x-ray tube current-time product (mAs) needed to obtain the desired x-ray detector radiation level. When the kV and mAs are selected by the operator, the desired x-ray detector radiation level is not always achieved. In one embodiment, the radiography computing device 18 may automatically select an appropriate kV and mAs when the x-ray focal spot and anti-scatter grid 3 are aligned for a desired digital x-ray detector radiation level using the patient's anatomy of interested selected by the operator and analysis of the test image.

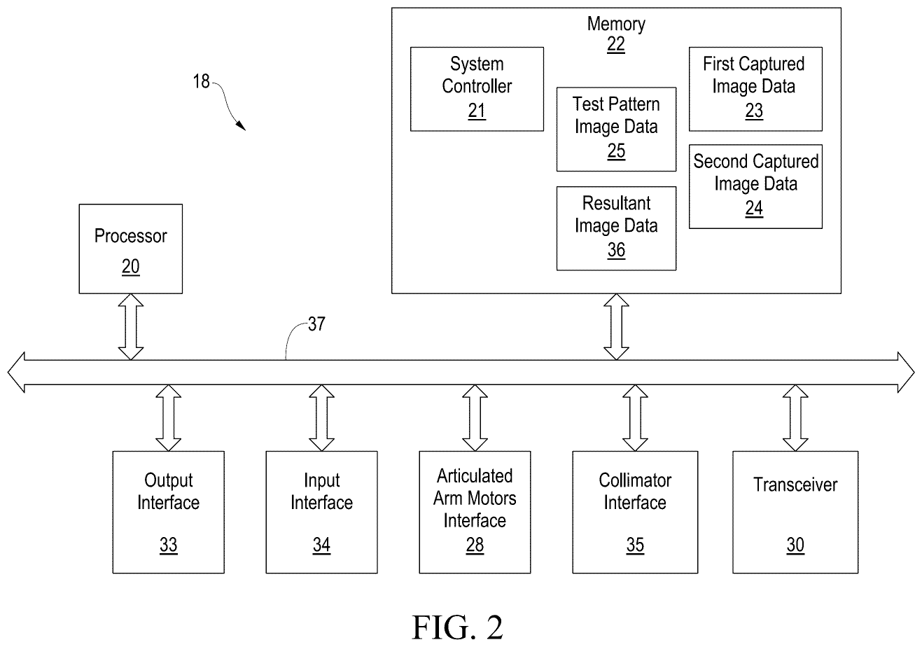

FIG. 2 is a block diagram of an exemplary radiography computing device 18 in accordance with an embodiment of the present disclosure. The exemplary radiography computing device 18 comprises a processor 20 and memory 22. Stored in memory 22 is a system controller 21. The system controller 21 controls the functionality of the radiography system 2 (FIG. 1).

Note that the system controller 21, when implemented in software, is stored, and transported on any computer-readable medium for use by or in connection with an instruction execution apparatus that can fetch and execute instructions. In the context of this document, a “computer-readable medium” can be any means that can contain or store a computer program for use by or in connection with an instruction execution apparatus.

The exemplary embodiment of the radiography computing device 18 depicted by FIG. 2 comprises at least one conventional processor 20, such as a digital signal processor (DSP) or a central processing unit (CPU), that communicates to and drives the other elements within the radiography computing device 18 via a local interface 37, which can include at least one bus. Further, the processor 20 is configured to execute instructions of software, such as instructions of the system controller 21.

An input interface 34, for example, atouchscreen, or keypad can be used to input data by an operator of the radiography system 2. An output interface 33, for example, a display device (e.g., a liquid crystal display (LCD)), can be used to output data indicative of information and images to the operator.

In addition, the radiography computing device 18 further comprises a transceiver 30. The transceiver 30 transmits and receives data wirelessly. In this regard, the transceiver receives data indicative of images captured by the digital x-ray detector 4 (FIG. 1). Further, during operation, first captured image data 23, second captured image data 24, test pattern image data 25, and resultant image data 36 are stored in memory 22.

The radiography computing device 18 further comprises an articulated arm motor(s) interface 28 for controlling movement of the articulated arm 20 (FIG. 1).

In one embodiment, the system controller 21 receives data indicative of a first image captured via the transceiver 30 from the digital x-ray detector 4 and stores the data received as first captured image data 23. The first captured image data 23 comprises data indicative of a test pattern image combined with data indicative of the patient's anatomy of interest. The second captured image data 24 comprises data indicative of only the patient's anatomy of interest.

Upon receipt of the first captured image data 23 and the second captured image data 24, the system controller 21 subtracts the second captured image data 24 from the first captured image data 23 to obtain only the test pattern image data 25. The system controller 21 analyzes the test pattern image data 25 to determine the digital x-ray detector's pixel location of the x-ray beam, the tilt of the digital x-ray detector 4 (FIG. 1) and the planes of the anti-scatter grid 3 (FIG. 1) relative to the central x-ray.

Note that subtraction of the second captured image data 24 from the first captured image data 23 to obtain only the test pattern image data 25 is merely an exemplary method for obtaining the test pattern image data 25. Other methods may be used in other embodiments.

Note that in one embodiment, an increased amount of power of the X-rays may be used to generate a single image. The radiography computing device 18 is further configured to determine the test pattern based upon the single image.

The radiography computing device 18 communicates to the operator, e.g., via the output interface 33, that the first captured image data 23 and the second captured image data 24 have been received. Under operator control, the radiography computing device 18 automatically actuates the system controller 21, which in turn transmits signals to the motors (not shown) associated with each degree of freedom of the articulated arm 20 and moves the source assembly 5 so that x-ray focal spot lies on the focal axis of the anti-scatter grid 3 and the central X-ray is centered on and orthogonal to the planes of the anti-scatter grid 3 and digital x-ray detector 4.

The system controller 21 transmits a signal via a collimator interface 35 to restrict the x-ray beam to the active area of the digital x-ray detector 4. When alignment of each degree of freedom is achieved and the x-ray beam is centered on and collimated to the digital x-ray detector 4, the system controller 21 notifies the operator, e.g., via the output interface 33. Upon notification, the operator selects the x-ray techniques for the exam and patient's habitus and initiates the x-ray exposure via the input interface 34. The x-ray generator 23 produces current in the x-ray tube (not shown) of the x-ray source assembly 5. The X-rays produced by the x-ray source assembly 5 expose the digital x-ray detector 4 activating the digital x-ray detector 4 to transmit data indicative of the image to the radiography computing device 18. The radiography computing device 18 receives data indicative of the resultant image and stores the data received as resultant image data 36 in memory 22. The system controller 21 processes and displays the resultant image data 36 to the display device 19 via the output interface 33.

In one embodiment, the system controller 21 directs the operator to sequentially move one or more degree of motion freedom at a time and the motion of each degree of freedom is locked when its desired location is achieved. When the alignments of all the degrees of freedom have been achieved and the x-ray beam collimated to the digital x-ray detector 4, the system controller 21 notifies the operator via the output interface 33.

FIG. 3A is an exemplary x-ray source assembly 5 and collimator 14 in accordance with an embodiment of the present disclosure. The exemplary x-ray source assembly 5 inherently has a focal spot 26.

The x-ray source assembly 5 is comprised of housing 22, collimator mounting plate 23, and x-ray tube and associated components (not shown) for performing an x-ray exposure. The housing 22 and housing mounting plate 23 are radiopaque except for aradiolucent window in the housing 22 and mounting plate 23 through which the X-rays pass. Coupled to the mounting plate 23 is the collimator 14. The collimator 14 is comprised of a housing 16 and movable radiopaque shutters 350,351,352 (not shown), and 353. The top of the housing 16 has openings (not shown) through which the x-ray beam passes and for coupling the collimator 14 to the collimator mounting plate 23. The remainder of the top of housing 16 is radiopaque and its sides are radiopaque. A bottom of the collimator housing 16 is radiolucent. The movable radiopaque shutters 350,351,352 (not shown) and 353 spatially restrict and define the span of the x-ray beam 39 in two dimensions emitted by the x-ray source assembly 5 and is incident on the patient 1 (FIG. 1) and digital x-ray detector 4. The radiopaque shutters 350, 351, 352 (not shown) and 353 can be moved manually or automatically.

Note that the X-ray collimator 14 comprises the pairs of moveable shutters 350,351 and 352 (FIG. 3B), 353. As shown in FIG. 3A, shutters 350 and 351 are shown on the left and right opposing one another, and only one shutter of the pair 352 (FIG. 3B) and 353, which is shutter 353 (the back shutter), is shown. The shutters are moveable toward and away from the central ray 28 (FIG. 3A).

The x-ray tube has a cathode (not shown), anode (not shown), and the focal spot 26. The focal spot 26 is the small area on the anode where X-rays originate. The x-ray source assembly's cathode-anode axis 27 is a line parallel to the plane of the mounting plate that passes through the cathode, anode, and focal spot 26. The central ray 28 of the x-ray beam is the line that passes through the focal spot 26 and is perpendicular to the plane of the mounting plate 23 and to the cathode-anode axis 27. In one embodiment the collimator 14 and spatially defined x-ray beam can rotate about the central ray 28.

In one embodiment, the collimator 14 has additional pairs of moveable shutters (not shown) closer to the focal spot of the X-ray tube. The additional pairs of moveable shutters are aligned with and move with shutters 350, 351, 352 (not shown), and 353.

In operation, the x-ray source assembly 5 emits X-rays. The X-rays enter the collimator 14. The radiopaque shutters 350, 351, 352, and 353 adjust the extent of the X-ray beam 39 (FIG. 3A) incident on the patient 1 (FIG. 1), anti-scatter grid 3, and digital X-ray detector 4 (FIG. 1).

Note that the focal spot 26 in the x-ray tube is two dimensional and state-of-the-art general radiography x-ray tubes typically have two focal spots, a large and small focal spot (not shown) with nominal sizes in one dimension of large focal spot ranging in size from 1.0 to 2.0 mm and the small spot from 0.3 to 1.0 mm.

The large x-ray tube focal spot can deliver greater x-ray power than the small focal spot, while the small focal spot images objects some distance above the x-ray detector with greater sharpness when less x-ray power is needed.

In one embodiment, the x-ray tube has a small focal spot size of 0.3 mm or smaller. In such an embodiment, the image captured would exhibit a sharper test pattern than would result with the larger focal spot. A sharper image of the test pattern permits more precise determination of the location of the x-ray focal spot relative to the x-ray image detector 4 and anti-scatter grid 3 than a less sharp image would.

FIG. 3B is a diagram of exemplary shutters 350-353 that may be arranged as shown in FIG. 3B relative to the each other. The diagram shown is a view looking down from the top of the collimator 14 when the shutters 350-353 are installed or positioned in the collimator 14. Each shutter350-353 is moveable inwards and outwards from a central point where the central ray 28 (FIG. 3A) is directed.

The left shutter 350 comprises a slot 354. In the embodiment shown, the slot 354 is rectangular. However, the slot 354 may be other shapes in other embodiments. Further, the right shutter 351 comprises a slot 355. In the embodiment shown, the slot 355 is rectangular. Additionally, the back shutter353 comprises a slot 356. In the embodiment shown, the slot 356 is rectangular. However, the slot 355 may be other shapes in other embodiments. The front shutter 352 comprises slots 357 and 358. In the embodiment shown, the slots 357 and 358 are rectangular. However, the slots 357 and 358 may be other shapes in other embodiments. The slots 357 and 358 are positioned on the front shutter 352 so that a central point between them is aligned with the slot 356 of the back shutter 353.

FIG. 3C is a diagram of an exemplary projected alignment image 359 resulting when X-rays pass through the collimator 14 having the shutters 350-353 installed therein. The projected alignment image comprises a rectangular (or square) outline. Note the projected alignment image will be rectangular/square in a plane perpendicular to the central ray. However, if the plane is not perpendicular to the central ray, the projected alignment image is an irregular quadrilateral. In the outline of the projected alignment image 359, there are five spaces where X-rays travel through the slots 354-358. These are shown on the projected alignment image 359 as voids 360-364.

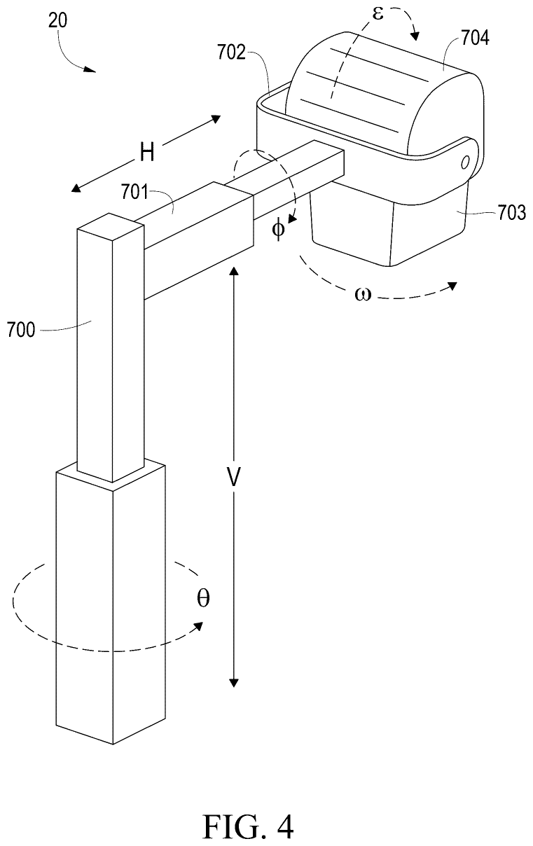

FIG. 4 is an exemplary articulating arm 20 in accordance with an embodiment of the present disclosure. The articulating arm 20 comprises a rotating vertical column 700. In this regard, the system controller 21 (FIG. 2) is configured to transmit signals to the articulating arm motor(s) (not shown) via the articulating arm motor(s) interface 28 (FIG. 2) to move the x-ray source assembly 704 vertically “V” and rotate the vertical column (and x-ray source assembly 704) about angle 8.

Further, the articulating arm 20 comprises a horizontal arm 701. In this regard, the system controller 21 is configured to transmit signals to the articulating arm motor(s) (not shown) via the articulating arm motor(s) interface 28 (FIG. 2) to move the horizontal arm 701 and the x-ray source assembly 704 horizontally “H.”

A gimbal 702 is rotatably coupled to the horizontal arm 701 via a bracket (not shown) that allows angle$ rotation of gimbal 702 (and x-ray source assembly 704) about the axis of the horizontal arm (not shown) and allows angle m rotation of the gimbal 702 (and x-ray source assembly 704) about an axis parallel to a central ray (not shown) exiting the x-ray source assembly 704. In this regard, the system controller 21 is configured to transmit signals to the articulating arm motor(s) (not shown) via the articulating arm motor(s) interface 28 (FIG. 2) to rotate the gimbal 702 about angles ¹.Dand _{OJ}.

The x-ray source assembly 704 is rotatable coupled to the gimbal 702 such that the x-ray source assembly 701 can rotate about angle c: about an x-ray tube cathode-anode axis. In this regard, the system controller 21 is configured to transmit signals to the articulating arm motor(s) (not shown) via the articulating arm motor(s) interface 28 (FIG. 2) to rotate the x-ray source assembly 704 about angle c:.

In one embodiment, the gimbal 702 is rotatably coupled to the horizontal arm 701 via a bracket (not shown) that allows angle$ rotation of the gimbal 702 (and x-ray source assembly 704 about the axis of the horizontal arm (not shown), and the collimator 14 is rotatably coupled to the x-ray source assembly 704 allowing angle _{OJ} rotation of the collimator 14 about the central ray (not shown). In this embodiment, the system controller 21 is configured to transmit signals to the articulating arm motor(s) (not shown) via the articulating arm motor(s) interface 28 (FIG. 2) to rotate gimbal 702 about angle$ and the collimator 703 about _{OJ}.

Note that as described above, the horizontal arm 701 and the vertical column 700 are telescoping. In this regard, the system controller 21 is configured to transmit signals to the articulating arm motor(s) (not shown) via the articulating arm motor(s) interface 28 (FIG. 2) to move the vertical column 700 in the “V” direction and the horizontal arm 701 in the “H” direction.

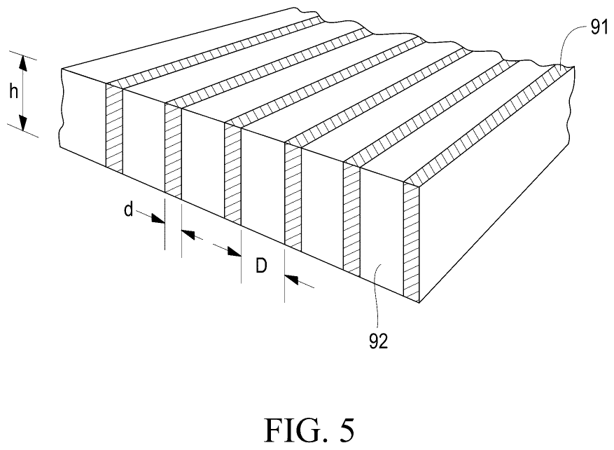

FIG. 5 is a diagram of a portion of an anti-scatter grid 3 (FIG. 1). The anti-scatter grid 3 comprises a plurality of radiopaque strips 91. The radiopaque strips 91 are often made of lead, which prevents X-rays from propagating through the lead strips 91.

The anti-scatter grid 3 further comprises a plurality of radiolucent interspace material 92 interposed between each lead strip 91. The radiolucent interspace material 92 is permeable by X-rays. In this regard, the radiolucent interspace material 92 does not block the X-rays and allows a majority of the X-rays to pass.

The grid ratio (h/D) is the ratio of the height of the lead strips 91 (h) to the distance (D) between them. The grid ratio (h/D) affects the scatter control efficiency of the grid. In this regard, the higher grid ratio anti-scatter grid 3, when aligned with the x-ray tube focal spot, reduces the scatter content and enhances the contrast of the x-ray image produced compared to an image produced with a low ratio grid.

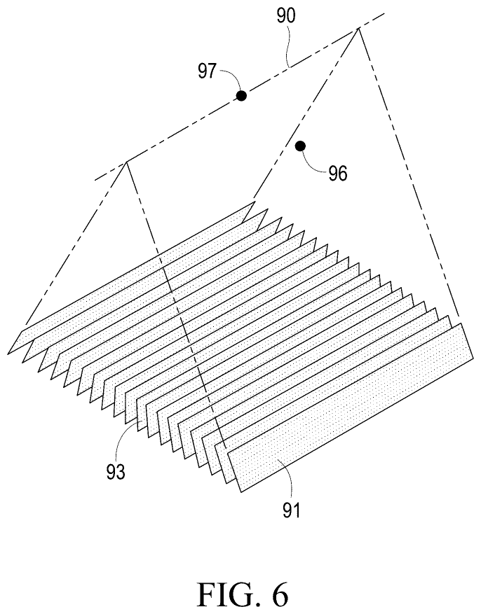

FIG. 6 is a diagram of a plurality of lead strips 91 making up a portion of the anti-scatter grid 3 (FIG. 1). The lead strips 91 are arranged such that each lead strip is angled inward toward the central lead strip 93, and the lead strips 91 of the anti-scatter grid 3 have a focal axis 90.

A misaligned focal spot anti-scatter grid position is denoted by 96. When the x-ray tube focal spot 26 (FIG. 4) is at position 96 it does not lie on the grid focal axis 90, is not aligned with the anti-scatter grid 3, and the central ray is not orthogonal to the plane of the anti-scatter grid. The centrally aligned focal spot anti-scatter grid position is denoted by 97. When the x-ray tube focal spot is at position 97 it lies on the grid focal axis 90 and the central ray 28 (FIG. 3A) is orthogonal to and centered on the anti-scatter grid 3 (FIG. 1), and is orthogonal to and centered on the digital x-ray detector 4 (FIG. 1). The image produced by exposure to the digital x-ray detector better reflects the anatomy of interest of the patient 1 (FIG. 1) than when the x-ray tube focal spot 26 is not aligned with the anti-scatter grid 3.

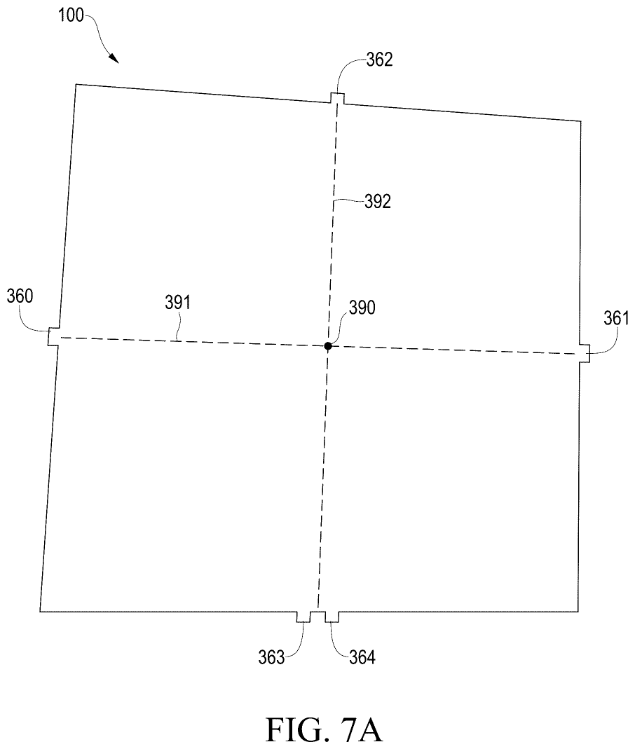

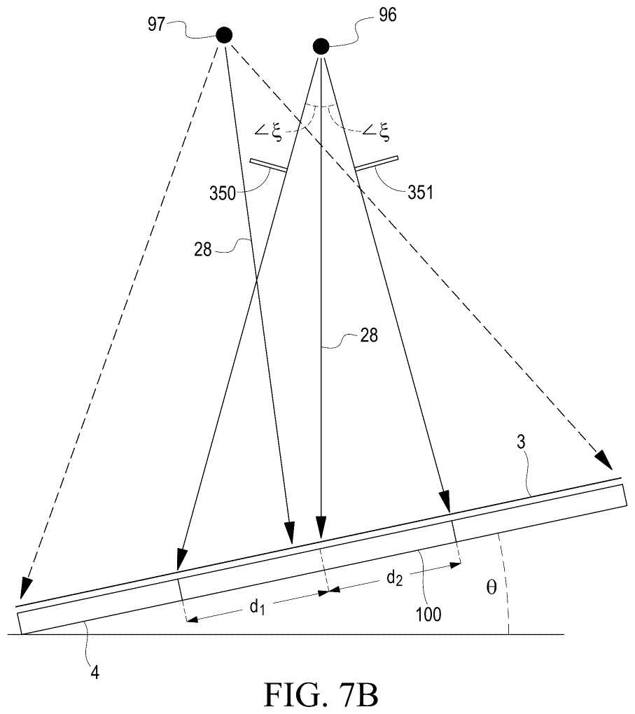

FIG. 7A is a diagram of the x-ray test image 100 obtained by detecting the projected x-ray alignment image 359 (FIG. 3C) by the x-ray detector in a plane tilted relative to a plane orthogonal to the central ray. The system controller 21 (FIG. 2) utilizes the location of voids 360-364 (FIG. 3C) to determine lines 390 and 391 in the test image. The point 390 at which lines 391 and 392 cross is the location of the central ray 28 (FIG. 3A) in the test image. Similarly, the system controller 21 uses the location of the voids 363 and 364 (FIG. 3C) to determine the location of the test image relative to the x-ray detector 4 (FIG. 1).

FIG. 7B is a diagram depicting the method whereby the system controller 21 (FIG. 2) uses the test image 100 to determine the location of the x-ray tube focal spot 26 (FIG. 4) relative to the location of the anti-scatter grid 3 (FIG. 1) and x-ray detector 4 (FIG. 1). Shown are shutters 350, 351 (FIG. 3B) of collimator 14 (FIG. 1). Not shown are shutters 352, 353. The collimator shutters are adjusted to a predetermined small field-of-view with angle between the edges of the x-ray beam and the central ray. A low dose X-ray is initiated and an alignment image 359 (FIG. 3C) is projected onto the digital x-ray detector 4. A second low dose X-ray is initiated with the collimator shutters adjusted to a second predetermined and slightly larger field-of-view to obtain a second alignment image. The system controller 21 subtracts the second image from the first image to obtain test image 100.

Shown are distances d1 and d2 determined from the test image 100 by the system controller 21 along line 391 (FIG. 7A). d1 to the left and d2 to the right of the central ray 28 location in the test image. The tilt angle 8 is a function of d1/d2.

$$δ = f ( {d}_{1} / {d}_{2} )$$

The tilt angle 8 increases as the d1/d2 ratio increases and the system controller 21 uses the ratio to determine the tilt angle 8. The following table illustrates the functional relationship of 8 on the d1/d2 ratio where is equal to 5 degrees.

| | 8 | di/d2 |
|---|---|---|
| 50 | 00 | 1.000000 |
| | 10 | 1.003059 |
| | 2.5° | 1.007669 |
| | 50 | 1.015427 |
| | 7.5° | 1.023305 |
| | 100 | 1.031337 |
| | 12.5° | 1.039559 |
| | 15° | 1.048011 |
| | 17.5° | 1.056735 |
| | 20° | 1.065781 |

Similarly, the system controller 21 determines tilt angle along line 392 (FIG. 7A).

Knowing the distance from the x-ray tube focal spot-to-projection image and projection image dimensions when it exits the collimator, the tilt of the planes of the anti-scatter grid and digital x-ray detector, the system controller 21 uses this information and test image 100 to determine the location and orientation of the anti-scatter grid 3 (FIG. 1) and digital x-ray image detector 4 (FIG. 1) relative to the movable x-ray source assembly focal spot 26.

When the location and orientation of the anti-scatter grid and digital x-ray detector is determined relative to the focal spot and moveable source assembly, the operator is notified. Under operator control of the x-ray source assembly 5 and x-ray tube focal spot 26 is automatically moved from misaligned x-ray tube focal position 96 to position 97. When the x-ray focal spot 26 is in position 97 it is aligned with the anti-scatter grid 3 and digital x-ray detector 4. It lies on the focal axis of the grid 90, the central X-ray 28 is orthogonal to the planes of the anti-scatter grid 3 and digital x-ray detector 4 and is centered on the anti-scatter grid 3 and digital x-ray detector 4.

In an alternative embodiment when the location and orientation of the anti-scatter grid and digital x-ray detector is determined relative to the focal spot and moveable source assembly, the operator is notified and the operator is directed to move one or more degrees of motion freedom of the x-ray source assembly 5 and x-ray tube focal spot 26 from misaligned x-ray tube focal position 96 to position 97. When a motion degree of freedom has reached the position where for that degree of freedom the x-ray focal spot 26 will be in position 97, that motion degree of freedom is locked. Subsequently when all motion degrees of freedom have been moved to their appropriate positions and locked, the x-ray tube focal spot 26 is aligned with the anti-scatter grid and digital x-ray detector. The focal spot lies on the focal axis of the grid 90, the central X-ray 28 is orthogonal to the planes of the anti-scatter grid and digital x-ray detector and is centered on the anti-scatter grid and digital x-ray detector.

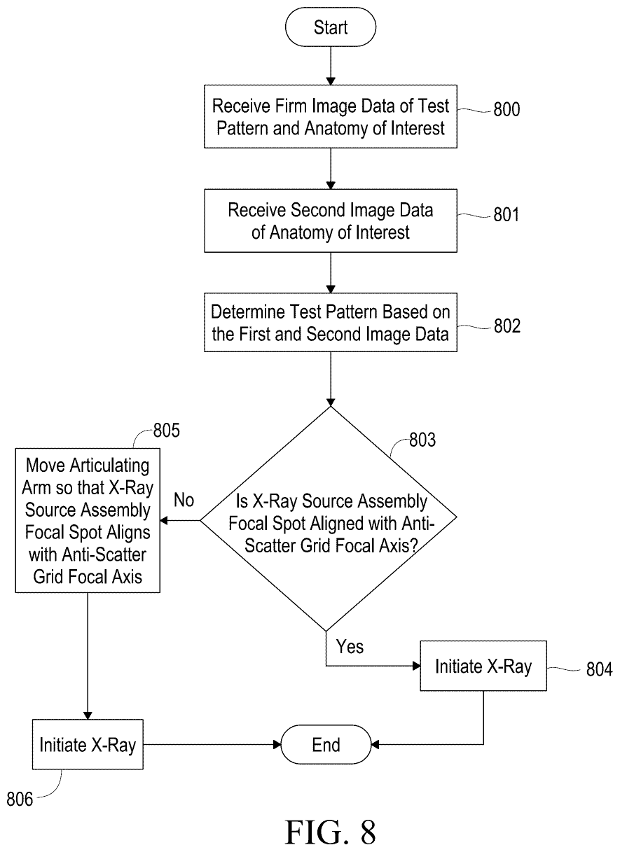

FIG. 8 is a flowchart of exemplary architecture and functionality of the mobile radiography system 2 (FIG. 1).

In step 800, the system controller 21 receives the first image indicative of the exposure resulting from the shutters 350-353 (FIG. 3B) and the anatomy of interest. In step 801, the system controller 21 receives the second slightly different image indicative of the anatomy of interest and the shutters 350-353 (FIG. 3B).

In step 802, the system controller 21 determines the test pattern image based on the first image and the second image. The system controller 21 analyzes the test image and determines the position of the x-ray focal spot 26 relative to the centrally aligned focal spot—grid position 97 (FIG. 7). If the x-ray focal spot is at position 96 (FIG. 7) and misaligned, the system controller 21 determines movement of the articulated arm 20 (FIG. 1) for positioning the x-ray focal spot at the centrally aligned position 97.

In step 803, the system controller 21 determines whether the x-ray source assembly focal spot 26 is at position 97 and is aligned with the anti-scatter grid focal axis 90. If they are aligned, the system controller 21 informs the operator to initiate an X-ray of the patient 1 (FIG. 1) in step 804. If the x-ray focal spot at position 96 (FIG. 7) is misaligned, the system controller 21 informs the operator.

When the x-ray focal spot and grid are misaligned under operator control in step 805, the system controller 21 moves the x-ray focal spot from misaligned position 96 (FIG. 7) and to centrally aligned position 97 (FIG. 7). When the x-ray focal spot is in position 97 the collimator shutters are adjusted so that the spatial extent of the X-ray beam is aligned with the X-ray detector. The operator is informed to initiate an X-ray of the patient 1 (FIG. 1) in step 806.

When the kV and mAs for the anatomy of interest is selected by the operator, the radiation level at the image detector is often less or more than the appropriate level. In one embodiment, using the anatomy of interest selected by the operator and the information in the test image (or images), the system controller 21 determines the kV and mAs to achieve an appropriate radiation level at the x-ray detector.

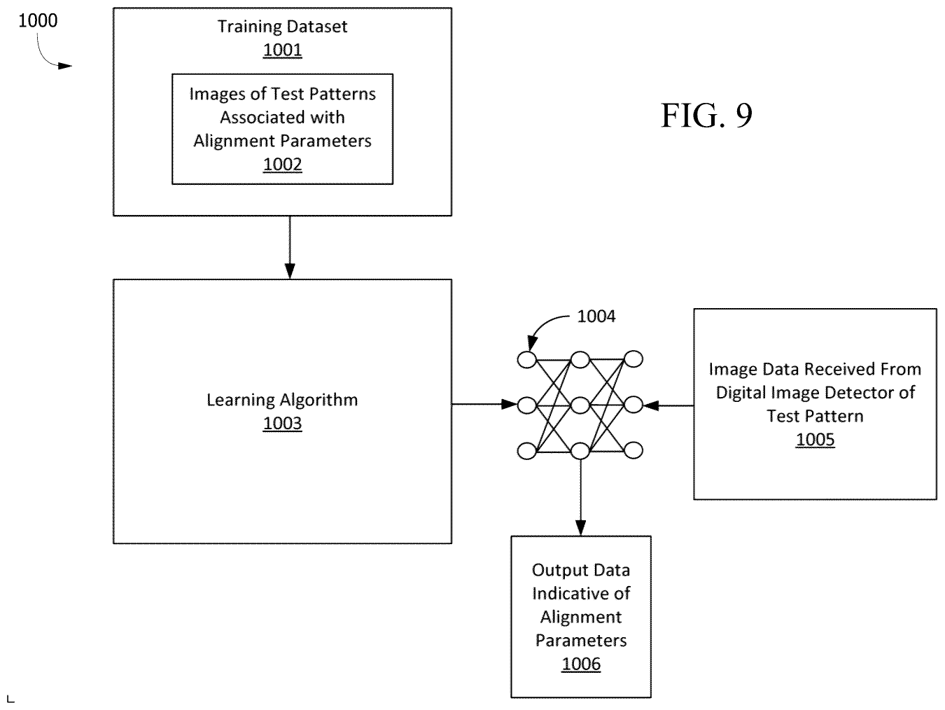

FIG. 9 is a block diagram of an exemplary artificial intelligence implementation 1000 for use in the radiography computing device 18 in accordance with an embodiment of the present disclosure. In this regard, the radiography computing device 18 may use ai-based image recognition to identify test images and determine orientation of the x-ray source assembly 5 (FIG. 1) relative to the digital x-ray detector 4 (FIG. 1).

The radiography computing device 18 having an artificial intelligence implementation will be trained using a dataset 1001. Training the radiography computing device 18 is teaching the radiography computing device 18 to properly interpret input images from the digital x-ray detector 4 (FIG. 1) and determine orientation so that the x-ray source assembly 5 may be aligned with the focal axis of the anti-scatter grid 3.

The dataset 1001 may comprise images of test patterns and associated with the images metadata that includes orientation parameters 1002. The radiography computing device 18 may be trained via machine learning. In one embodiment, the machine learning is supervised learning in which the radiography computing device 18 builds a model via a machine learning algorithm 1003. In this regard, the radiography computing device 18 uses the image and parameter data 1002 to find a model that minimizes incorrect outcomes, i.e., analyzing an input image and incorrectly determining the orientation of the digital x-ray detector 4 in relation to the x-ray source assembly 5.

A model 1004 is generated by using the learning algorithm 1003. The model 1004, when used during operation of the radiography computing device 18, analyzes datasets (i.e., input images) to find patterns and predict the orientation of the x-ray source assembly 5 relative to the digital x-ray detector 4. Based upon the orientation predicted, the radiography computing device 18 determines how to move the x-ray source assembly 5 to align the focal array of the anti-scatter grid 3 with the x-ray source assembly 5.

Note that there are image recognition algorithms that may be used to generate the model 1004 by the radiography computing device 18. For example, SIFT (scale-invariant feature transform), SURF (speeded up robust features), PCA (Principal component analysis), and LDA (linear discriminant analysis). Note that these algorithms are exemplary, and other algorithms may be used in other embodiments.

Note that there are models that may be used in an embodiment of the radiography computing device 18 for image classification. For example, the model 1004 may be a VOLOv5 model, Vision Transformer model or Resnet34 model. Other models may be used in other embodiments. Also, convolutional neural networks (CNNs) may be used as the model 1004.

In operation, an image captured by the digital x-ray detector 4 is input into the model 1004. Based upon the input, the model 1004 outputs data indicative of alignment parameters. Thus, the radiography computing device 18 may use the alignment parameters 1006 to determine the direction and/or angles to move each of the degrees of freedom of the articulated arm 20 to centrally align the focal spot 26 (FIG. 4) of the x-ray source assembly 5 (FIG. 1) with the focal axis 90 (FIG. 7) of the anti-scatter grid 3 and collimate the spatial extent (length and width) of the x-ray beam to the digital x-ray detector 4.

What I claim is:

1. A mobile radiography system, comprising:
an x-ray source assembly configured to activate a digital x-ray detector through X-rays striking the digital x-ray detector;
one or more shutters installed in a collimator in a stream of the X-rays configured to project an alignment image onto the digital x-ray detector;
a processor configured for receiving data indicative of the alignment image and the anatomy of interest, generating a first digital image indicative of the alignment image and the anatomy of interest, the processor further configured for analyzing the first image and determining if a focal spot of the x-ray source assembly is aligned with a focal axis of an anti-scatter grid and the digital x-ray detector based on the first digital image, and when the focal spot of the x-ray source assembly is misaligned with the focal axis of the anti-scatter grid and center of the digital x-ray detector, the processor further configured for aligning the focal spot of the x-ray source assembly with the focal axis of the anti-scatter grid and the center of the digital x-ray detector or the processor is further configured for providing instructions to an operator for manually aligning the focal spot of the x-ray source assembly with the focal axis of the anti-scatter grid and the center of the digital x-ray detector.

2. The mobile radiography system of claim 1, wherein the one or more shutters comprises at least one pair of shutters moveably affixed on a left and right side relative to the stream of X-rays.

3. The mobile radiography system of claim 2, wherein each shutter of the pair of shutters comprises a fiducial marker oriented open toward a central X-ray in the X-rays.

4. The mobile radiography system of claim 3, wherein the fiducial marker is centrally located on their respective shutters.

5. The mobile radiography system of claim 4, wherein the fiducial markers are rectangular.

6. The mobile radiography system of claim 1, wherein the one or more shutters comprises at least one pair of shutters moveably affixed on a back and a front side relative to the stream of X-rays.

7. The mobile radiography system of claim 5, wherein the shutter affixed on the back side comprises a fiducial marker oriented open toward a central X-ray in the X-rays.

8. The mobile radiography system of claim 6, wherein the fiducial marker on the shutter is moveably affixed to the back side is centrally located.

9. The mobile radiography system of claim 8, wherein the shutter affixed on the front side comprises at least two fiducial markers opposing the fiducial marker on the shutter moveably affixed to the back side.

10. The mobile radiography system of claim 9, wherein each of the two fiducial markers on the shutter moveably affixed to the front side are aligned equidistant from the opposing fiducial marker on the shutter moveably affixed to the back side.

11. The mobile radiography system of claim 1, wherein the processor is further configured for receiving a data indicative of the anatomy of interest and generate a second image of the data indicative of the anatomy of interest.

12. The mobile radiography system of claim 11, wherein the processor is further configured to for determining the alignment image by subtracting the second image from the first image.

13. The mobile radiography system of claim 1, wherein the radiography computing device may automatically selects an appropriate kV and mAs when the x-ray focal spot and anti-scatter grid are aligned.

14. The mobile radiography system of claim 1, wherein the processor is configured for receiving data indicative of a location of a location of the x-ray source assembly and automatically locking at least one degree of freedom based upon the data indicative of the location.

15. A mobile radiography method, comprising:
activating, by an x-ray source assembly, a digital x-ray detector through striking the digital x-ray detector with X-rays;
imaging, by one or more shutters in a collimator in a stream of the X-rays, an alignment image on the digital x-ray detector;
receiving, by a processor, data indicative of the alignment and an anatomy of interest;
generating, by a processor, a first image from the data indicative of the alignment image and the anatomy of interest;
analyzing, by a processor, the first image;
determining, by a processor, if a focal spot of the x-ray source assembly is aligned with a focal axis of an anti-scatter grid and the digital x-ray detector based on the data indicative of the alignment image; and
aligning, by a processor, the focal spot of the x-ray source assembly with the focal axis of the anti-scatter grid and the center of the digital x-ray detector or the processor is further configured for providing instructions to an operator for manually aligning the focal spot of the x-ray source assembly with the focal axis of the anti-scatter grid and the center of the digital x-ray detector when the focal spot of the x-ray source assembly is misaligned with the focal axis of the anti-scatter grid and center of the digital x-ray detector.

16. The mobile radiography method of claim 15, wherein the one or more shutters comprises at least one pair of shutters moveably affixed on a left and right side relative to the stream of X-rays, wherein each shutter of the pair of shutters comprises a fiducial marker oriented open toward a central X-ray in the X-rays and the fiducial markers are centrally located on their respective shutters, further comprising determining a location of the x-ray source based upon the fiducial markers.

17. The mobile radiography system of claim 16, wherein the one or more shutters comprises at least one pair of shutters moveably affixed on a back and a front side relative to the stream of X-rays, the shutters affixed to the front side and the back side each comprising a fiducial marker, further comprising determining, by the processor, a location of the x-ray source based upon the fiducial markers.

18. The mobile radiography method of claim 15, further comprising receiving, by the processor, data indicative of the anatomy of interest and generating a second image of the data indicative of the anatomy of interest.

19. The mobile radiography method of claim 18, further comprising determining, by the processor, the alignment image by subtracting the second image from the first image.

20. The mobile radiography method of claim 15, further comprising automatically selecting, by the processor, an appropriate kV and mAs when the x-ray focal spot and anti-scatter grid are aligned.

21. The mobile radiography method of claim 15, further comprising receiving data indicative of a location of a location of the x-ray source assembly and automatically locking, by the processor, at least one degree of freedom based upon the data indicative of the location.
